(19) United States
(12) Patent Application Publication
(10) Pub. No.: US 2024/0008743 A1
(43) Pub. Date: Jan. 11, 2024

(54) VENTILATOR COMPRISING A DEVICE FOR IDENTIFYING MAGNETIC FIELDS AND A DEVICE FOR ALERTING UPON IDENTIFICATION OF A MAGNETIC FIELD

(71) Applicant: Loewenstein Medical Technology S.A., Luxembourg (LU)

(72) Inventors: Lukas Hoehle, Frankfurt am Main (DE); Peter Kremeier, Karlsruhe (DE)

(21) Appl. No.: 18/346,944

(22) Filed: Jul. 5, 2023

(30) Foreign Application Priority Data
Jul. 7, 2022 (DE) 102022116983.9

(51) Int. Cl. A61B 5/00; A61B 5/055; G01R 33/02

(52) U.S. Cl. CPC A61B 5/0036; A61B 5/055; G01R 33/02; A61M 16/0003

(57) ABSTRACT

A ventilator comprises an identification device that is configured and designed to identify at least one magnetic field M.

### CROSS-REFERENCE TO RELATED APPLICATIONS

The present application claims priority under 35 U.S.C. § 119 of German Patent Application No. 102022116983.9 filed Jul. 7, 2022, the entire disclosure of which is expressly incorporated by reference herein.

### BACKGROUND OF THE INVENTION

### 1. Field of the Invention

The present invention relates to a ventilator comprising a device for identifying magnetic fields and a device for alerting upon identification of a magnetic field.

### 2. Discussion of Background Information

In addition to the treatment of sleeping illnesses and for respiratory assistance, ventilators are also used in the clinical setting for ventilation. Ventilators are used and required in many areas in the clinical setting, in particular upon use due to respiratory diseases.

Magnetic resonance tomographs (MRT)—also designated as magnetic resonance imagers or MRI—offer an imaging diagnostic method for generating detailed sectional images of tissue and organs in the clinical setting. MRTs generate very strong magnetic fields and alternating magnetic fields in the radiofrequency range. Depending on the design, the MRTs are differentiated into closed MRT systems and open MRT systems.

Permanent magnets or conventional electromagnets can be used for weak magnetic fields up to approximately 0.5 tesla flux density. Superconducting magnetic coils are used for stronger fields. Since the magnetic flux density has a direct effect on the signal quality of the detected data, a trend toward ever higher flux densities can be seen in medicine. In human medicine, the flux density for diagnostic purposes is presently typically at 1.5 T to 3.0 T. In recent years, ever higher flux densities of 7 tesla or more have been researched, so-called ultrahigh field systems.

A use of ventilators in conjunction with MRTs can result in interactions and disturbances of the ventilator and destruction of individual critical components of the ventilator.

In view of the foregoing, it would be advantageous to have available a ventilator which ensures safe operation of the ventilator.

### SUMMARY OF THE INVENTION

The invention provides a ventilator which is characterized in that the ventilator comprises an identification device that is configured and designed to identify at least one magnetic field.

In some embodiments, the ventilator is characterized in that the identification device is at least one sensor or comprises at least one sensor that is configured and designed to detect magnetic flux densities and/or magnetic field strengths to identify the magnetic field.

In some embodiments, the ventilator is characterized in that the sensor is a magnetic field sensor that is configured and designed to measure the magnetic field in three axes.

In some embodiments, the ventilator is characterized in that at least one limiting value is stored in the identification device, and in that the identification device is configured and designed to identify a magnetic field when the magnetic flux density or a statistical value of the magnetic flux density exceeds the limiting value.

In some embodiments, the ventilator is characterized in that the identification device is configured and designed to detect the magnetic flux density once or continuously or cyclically at time intervals.

In some embodiments, the ventilator is characterized in that the time intervals repeat regularly and/or irregularly.

In some embodiments, the ventilator is characterized in that the time intervals are constant or are ascertained and dynamically adapted in operation of the ventilator.

In some embodiments, the ventilator is characterized in that the identification device detects the magnetic flux density cyclically about every 60 seconds or more often, preferably about every 10 seconds or more often, particularly preferably about every 5 seconds or more often.

In some embodiments, the ventilator is characterized in that the identification device detects the magnetic flux density about every 4 seconds or more rarely, as long as the limiting value is not exceeded.

In some embodiments, the ventilator is characterized in that the identification device detects the magnetic flux density cyclically about every 4 seconds or more frequently, particularly preferably about every 1 second or more frequently, particularly preferably about every 500 milliseconds or more frequently, if the limiting value is exceeded.

In some embodiments, the ventilator is characterized in that the identification device detects the magnetic flux density about every 100 milliseconds if the limiting value is exceeded.

In some embodiments, the ventilator is characterized in that the identification device detects the magnetic flux density in a manner dynamically adapted to the magnetic field strength if the limiting value is exceeded.

In some embodiments, the ventilator is characterized in that the ventilator comprises a signal device that is configured and designed to emit at least one signal.

In some embodiments, the ventilator is characterized in that the identification device is configured and designed to control the signal device.

In some embodiments, the ventilator is characterized in that the signal device comprises an optical signal generator and/or an acoustic signal generator.

In some embodiments, the ventilator is characterized in that the identification device is configured and designed to identify an area in which the values of the magnetic flux density are equal to or less than the limiting value as a noncritical zone.

In some embodiments, the ventilator is characterized in that the limiting value is in a range from about 500 μT to about 5 mT, particularly preferably in a range from about 1 mT to about 4 mT.

In some embodiments, the ventilator is characterized in that the limiting value is 3 mT.

In some embodiments, the ventilator is characterized in that one or more additional limiting values are stored in the identification device, on the basis of which the identification device identifies two or more zones of the magnetic field.

In some embodiments, the ventilator is characterized in that a third limiting value is stored in the identification device and in that the identification device is configured and designed to identify an area in which the values of the magnetic flux density are higher than the third limiting value as a critical zone and/or collision zone.

In some embodiments, the ventilator is characterized in that the third limiting value is in a range from about 3 mT to about 100 mT, particularly preferably in a range from about 20 mT to about 70 mT.

In some embodiments, the ventilator is characterized in that the third limiting value is about mT.

In some embodiments, the ventilator is characterized in that a threshold value is stored in the identification device and in that the identification device is configured and designed to identify an area in which the values of the magnetic flux density are higher than the third limiting value and the threshold value is exceeded as a collision zone.

In some embodiments, the ventilator is characterized in that a second limiting value is stored in the identification device and in that the identification device is configured and designed to identify an area in which the values of the magnetic flux density are higher than the second limiting value and equal to or less than the third limiting value as a warning zone.

In some embodiments, the ventilator is characterized in that the second limiting value is in a range from about 3 mT to about 50 mT, particularly preferably in a range from about 10 mT to about 40 mT.

In some embodiments, the ventilator is characterized in that the second limiting value is about 20 mT.

In some embodiments, the ventilator is characterized in that the identification device is configured and designed to identify an area in which the values of the magnetic flux density are higher than the limiting value and equal to or less than the second limiting value as an operating zone.

In some embodiments, the ventilator is characterized in that the identification device activates the signal device on the basis of the identification of the various zones of the magnetic field in such a way that individual optical signals and/or acoustic signals are output depending on the zone.

In some embodiments, the ventilator is characterized in that the optical signals and/or the acoustic signals are output increasingly more intensely with increasing strength of the magnetic field.

In some embodiments, the ventilator is characterized in that the optical signals and/or the acoustic signals are manually or automatically adaptable.

In some embodiments, the ventilator is characterized in that the optical signal and/or the acoustic signal is automatically adapted if the identification device identifies that one of the limiting values is exceeded or fallen below.

In some embodiments, the ventilator is characterized in that the optical signal and/or the acoustic signal is automatically adapted if the identification device identifies that the threshold value is exceeded or fallen below.

In some embodiments, the ventilator is characterized in that the optical signals and/or the acoustic signals can be ended manually or automatically.

In some embodiments, the ventilator is characterized in that the optical signal and/or the acoustic signal which is output when the ventilator is in the collision zone can be ended only after critical components of the ventilator have been checked and/or exchanged.

In some embodiments, the ventilator is characterized in that an optical signal is output when the ventilator is in the noncritical zone, wherein the optical signal is output, for example, in the form of an intermittently flashing lighted green LED light.

In some embodiments, the ventilator is characterized in that an optical signal is output when the ventilator is in the operating zone, wherein the optical signal is output, for example, in the form of a flashing lighted green LED light.

In some embodiments, the ventilator is characterized in that an optical signal and an acoustic signal are output when the ventilator is in the warning zone, wherein the optical signal is output, for example, in the form of a flashing lighted yellow LED light and the acoustic signal is output, for example, in the form of an intermittent warning tone.

In some embodiments, the ventilator is characterized in that an optical signal and an acoustic signal are output when the ventilator is in the critical zone, wherein the optical signal is output, for example, in the form of a flashing lighted red LED light and the acoustic signal is output, for example, in the form of a continuous tone.

In some embodiments, the ventilator is characterized in that an optical signal and an acoustic signal are output when the ventilator is in the collision zone, wherein the optical signal is output, for example, in the form of a continuously lighted red LED light and the acoustic signal is output, for example, in the form of a continuous siren tone.

### BRIEF DESCRIPTION OF THE DRAWINGS

A ventilator according to the invention is described in the following exemplary embodiments. Further features and advantages of the present invention will become clear in the following descriptions of exemplary embodiments on the basis of the figures. The invention is not restricted to the illustrated exemplary embodiments.

Exemplary embodiments of the ventilator according to the invention are shown in the drawings, in which:

FIG. 1 shows a schematic illustration of a ventilator according to the invention in a perspective view.

FIG. 2 shows a schematic illustration of the magnetic field which originates from an MRT.

FIG. 3 shows a schematic illustration of different exemplary zones of the magnetic field which can be identified by the ventilator.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

A ventilator 10 is to be understood in the meaning of the invention as all devices which assist a patient or other user in natural breathing and/or take over the breathing of a user or patient and/or are used for respiratory treatment and/or affect the breathing of a user or patient in another way. This includes, for example, but not exclusively, ventilators for clinical or home applications, respiratory therapy devices, CPAP, APAP, and BiLevel devices, high flow therapy devices, narcosis or anesthesia devices, clinical, nonclinical, or emergency ventilators, oxygen- (O₂—) supplying devices, diagnostic systems, and coughing therapy devices or coughing machines. Ventilators can also be understood as diagnostic devices for respiratory treatment. Diagnostic devices can be used in general for detecting medical and/or breathing-related parameters of a living being. These also include devices which can detect and optionally process medical parameters of patients in combination with breathing or exclusively relating to breathing.

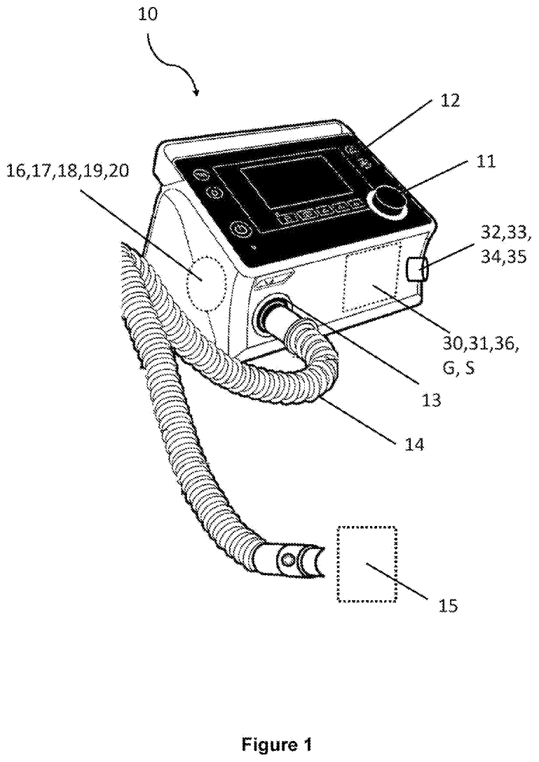

FIG. 1 shows a schematic illustration of a ventilator 10 according to the invention in a perspective view.

The ventilator 10 can be equipped with an operating device 11 and with a display device 12. The operation can take place at least partially via a touch-sensitive surface of the display device 120. Operation can also take place via mechanical switching elements such as knobs, buttons, and the like.

The ventilator 10 includes an interface 13 for coupling a hose system 14 for ventilation, respiratory assistance, respiratory therapy, diagnosis, and/or cough assistance. A patient interface 15 (not shown in more detail here) can be connected to the hose system 14. A patient interface 15 is to be understood in the meaning of the invention as any peripheral device which is designed for interaction with a living being. In particular, the patient interface 15 is designed for treatment and/or diagnostic purposes in conjunction with the ventilator 10. The patient interface 15 can be designed as a breathing mask. This includes, for example, but not exclusively, nose masks, nose cushion masks, nasal cannulas or oxygen cannulas, full face or total face masks, and tracheal tubes or cannulas.

The ventilator 10 is equipped with at least one fan device 16 (housed in the device interior, and so not visible here) according to the invention, using which a respiratory airflow for ventilation, respiratory assistance, respiratory therapy, and/or cough assistance is generated.

The fan device 16 can be or comprise a respiratory gas source. The fan device 16 can in some embodiments be or comprise a blower. The fan device 16 can in some embodiments also simply be or comprise a pressurized gas source. The respiratory airflow generated by means of the fan device 16 is supplied via the hose system 14 and the patient interface 15 to the patient or user (not shown). In the meaning of the invention, respiratory air comprises any fluid, respiratory gas, and/or gas mixture which is suitable and can be used for ventilation, breathing, and/or respiratory therapy.

To operate the fan device 16, the ventilator 10 includes an electrical drive 17 and at least one energy source 18. The ventilator 10 can be supplied with energy via a mains plug and alternatively or additionally via accumulators arranged in the device interior. The ventilator can thus be operated in mains operation and/or in accumulator operation.

The ventilator 10 can comprise a control device 19 and at least one storage device 20. The control device 19 can be configured and designed, inter alia, to control the ventilator 10. The control device 19 can be configured and designed, inter alia, to activate the fan device 16.

Thus, for example, respiratory gas pressure and/or flow and/or volume can be controlled. For example, the control device 19 sets a specific speed of the fan device 16 in dependence on the treatment specifications. The storage device 20 can be configured and designed, inter alia, to store ventilation-relevant data such as ventilation settings, ventilation statistics, ventilation histories, and the like. The storage device 20 can be or comprise, for example, at least one hard drive and/or at least one memory card and/or similar storage media.

The ventilator 10 according to the invention includes an identification device 30. The identification device 30 is configured and designed to detect and identify magnetic fields M. At least one limiting value G1 can be stored in the identification device 30. The identification device 30 can identify the presence of a magnetic field M, which is above the value of Earth's natural magnetic field, on the basis of the limiting value G1.

The identification device 30 can be configured and designed to detect a change of the electrical resistance which arises due to a magnetic field M having a magnetic flux density. For this purpose, the identification device 30 can be at least one sensor or comprise at least one sensor. The sensor is configured and designed to detect at least one magnetic flux density and/or one magnetic field strength and/or one magnetic flux and/or one magnetic field M. The sensor is a sensor selected from a magnetometer, a magnetic field sensor, a Hall sensor, a Tesla sensor, a Gauss sensor, a Reed sensor, a measurement coil.

In one specific embodiment, the sensor is a magnetic field sensor which is configured and designed to measure the magnetic field in three axes (x, y, z). The magnetic field sensor can be constructed with double redundancy and can detect the magnetic field acting on the ventilator, for example, at at least one point in the housing. The sensor preferably detects the magnetic field acting on the ventilator at multiple points in the housing, by way of example. In one specific embodiment, the sensor detects the magnetic field acting on the ventilator at at least two points in the housing, by way of example.

The identification device 30 can comprise at least one exposure counter function 36. The identification device 30 preferably comprises at least one sensor and an exposure counter function 36.

The identification device 30 can be configured and designed to identify a magnetic field M in that the magnetic flux density or a statistical value of the magnetic flux density, for example a mean value, is above the limiting value G1.

The identification device 30 is configured and designed to identify magnetic fields M once or continuously or at predefined points in time. For example, magnetic fields M can be cyclically detected at predefined points in time. The time intervals in which a cyclically repeating detection of the magnetic field M can take place can be regular or irregular. The detection of the magnetic field can in particular be dynamically adapted.

A cyclic detection and identification of magnetic fields M at specific points in time offers the advantage of being power-saving. Power-saving operation is required in particular if the ventilator 10 is removed from the power grid and is operated by the accumulator. The cyclic identification of magnetic fields M can extend the accumulator runtime. Smaller accumulators can therefore also be used, so that weight and size of the ventilator 10 can be reduced.

The time interval or the distance between the points in time at which magnetic fields M are identified can be stored in the ventilator 10, for example in the identification device 30 itself or in the storage device 20. The distance between the points in time at which magnetic fields M are identified can be constant. In preferred exemplary embodiments, the distance between the points in time at which magnetic fields M are identified can be ascertained and adapted in operation of the ventilator 10. The time interval can also be manually set and/or changed via the operating device 11.

The intervals between the points in time at which magnetic fields M are identified can be equal or different in mains operation and in accumulator operation. For example, the intervals between the points in time in mains operation can be selected to be less than in accumulator operation.

The identification device 30 can in some embodiments continuously detect magnetic fields M. In preferred embodiments, the identification device 30 can detect magnetic fields M cyclically at specific points in time. The time interval for the detection of magnetic fields can be milliseconds (ms), seconds (s), minutes (m), or else hours.

In some exemplary embodiments, the identification device 30 can detect magnetic fields M cyclically about every 500 ms or more frequently, particularly preferably about every 200 ms or more frequently. For example, the identification device 30 can cyclically detect magnetic fields M about every 100 ms.

A detection in the millisecond range can be possible and noncritical in particular if the ventilator 10 operates in mains operation.

A detection in the millisecond range can in particular also be advantageous if the identification device 30 identifies that the device is located in a magnetic field M relevant or critical for the ventilator 10. The identification device 30 can then be configured to carry out a magnetic field detection in the millisecond range. As soon the identification device 30 identifies that the ventilator 10 is in a magnetic field M having a magnetic flux density equal to or less than the limiting value G1, the measurement of the identification device 30 can take place more rarely, for example in the second range. The ventilator 10 can thus be operated in an energy-saving mode.

In some exemplary embodiments, the identification device 30 can detect magnetic fields M cyclically about every 1 s or less, in particular about every 2 s or less. For example, the identification device 30 can detect magnetic fields M cyclically about every 4 s.

A detection in the second range can be advantageous in particular if the ventilator 10 operates in accumulator operation. A detection in the second range can be power-saving and still offer a sufficient level of safety due to a rapid detection of magnetic fields M.

A detection in the second range can also be advantageous in particular if the identification device 30 identifies that the device is located in a magnetic field M noncritical for the ventilator 10. The identification device 30 can then be configured to carry out a magnetic field detection in the second range. As soon as the identification device 30 identifies that the ventilator 10 is in a magnetic field M having a magnetic flux density above the limiting value G1, the measurement of the identification device 30 can take place more frequently, for example in the millisecond range.

It is also conceivable that the identification device 30 detects magnetic fields M more rarely than about every 4 s, for example in minute cycles or else only in hour cycles or, for example, once a day. In some exemplary embodiments, the magnetic fields M can, for example, only be detected upon startup of the ventilator 10.

The ventilator 10 according to the invention can include at least one signal device 31. The identification device 30 is configured and designed to control the signal device 31. The signal device 31 is configured and designed to trigger and/or emit at least one signal and/or one alarm based on the data of the identification device 30. The signal device 31 can comprise an optical signal generator 32 and/or an acoustic signal generator 34.

The optical signal generator 32 can emit an optical signal 33. The optical signal 33 can take place in the form of light and/or color outputs and/or some other type of display on the ventilator 10. For this purpose, at least one lighting means can be integrated on or in the ventilator 10. For example, at least one LED light can be integrated in the ventilator 10, which can emit light in different colors and/or intensities. The optical signal 33 can take place, for example, in that at least one LED light emits light signals. For example, the LED light can regularly or irregularly flash, flicker, continuously blink, continuously light up, or emit similar signals. The optical signal 33 can also take place, for example, in that the at least one LED light emits color signals. For example, the LED light can emit color signals in the traffic signal colors green, yellow, red. Other colors are also conceivable.

The optical signal 33 can alternatively or additionally also be generated as a display in the form of at least one notification and/or an action instruction in the operating panel of the ventilator 10.

The acoustic signal generator 34 can emit an acoustic signal 35. The acoustic signal 35 can take place in the form of tones, tone sequences, sirens, speech outputs, or the like. The volume of the acoustic signal 35 can be at one level, increasing, or alternately increasing and decreasing. For the purpose of acoustic signal generation, at least one loudspeaker can be integrated on or in the ventilator 10.

The acoustic signal 35 can take place, for example, such that at least one loudspeaker emits acoustic signals. For example, the loudspeaker can emit at least one warning tone and/or alarm. The loudspeaker can emit intermittent individual tones, continuous warning tones, warning tones in differing volume and frequency, spoken warning notifications, or the like. The acoustic signal 35 can also be output in the form of a vibration alarm.

A change and/or ending of the optical signal 33 and/or of the acoustic signal 35 can take place manually or automatically.

For example, the optical signal 33 and/or the acoustic signal 35 can be changed or ended automatically by the ventilator 10 when the identification device 30 identifies exceeding or falling below a limiting value G1, G2, G3.

In some embodiments, the optical signal 33 and/or the acoustic signal 35 can also be carried out manually by a person. For this purpose, safety mechanisms can be stored in the signal device 31 which can regulate the access to the signals 33, 35. For example, access to the signal device 31 can be password-protected so that only authorized persons have access to the signal device 31 and the change and/or ending of the signal 33, 35. Alternatively or additionally, it can also be provided that at least one action has to be carried out before the signal 33, 35 is turned off. For example, the signal device 31 can be configured in such a way that a shutdown of the signal 33, 35 can only take place if a testing plan has been processed and/or critical components have been checked and/or exchanged. It is conceivable in this case that the acoustic signal 35 can be switched off without or with fewer safety mechanisms than the optical signal 33 in order to facilitate the handling of the device.

Critical components in the meaning of the invention are considered to be any components relevant for ventilation or respiratory assistance which can be changed and/or disturbed and/or destroyed by a magnetic field M. These components can be component parts of the ventilator itself or component parts which are connected to the ventilator 10, such as the hose system 14 or the patient interface 15, for example. Measurement instruments, diagnostic devices, valves, sensors, or the like used in conjunction with the ventilator 10 can also be considered to be critical components in the meaning of the invention.

Critical components of the ventilator 10 can be, for example, the one or more storage device and/or the drive 17. Critical components of the ventilator 10 can also be, for example, solenoid valves, voltage converters, or the like. Components which have inductive elements, such as actuators (voice coil actuators) or valves, are particularly critical in this case. Critical components can also be circuit parts having inductive elements such as DC/DC converters and/or AC/DC converters. Critical components can also be sensors, for example, which are based on (para-)magnetic effects. Such components react sensitively to magnetic radiation and can be changed and/or damaged and/or destroyed thereby.

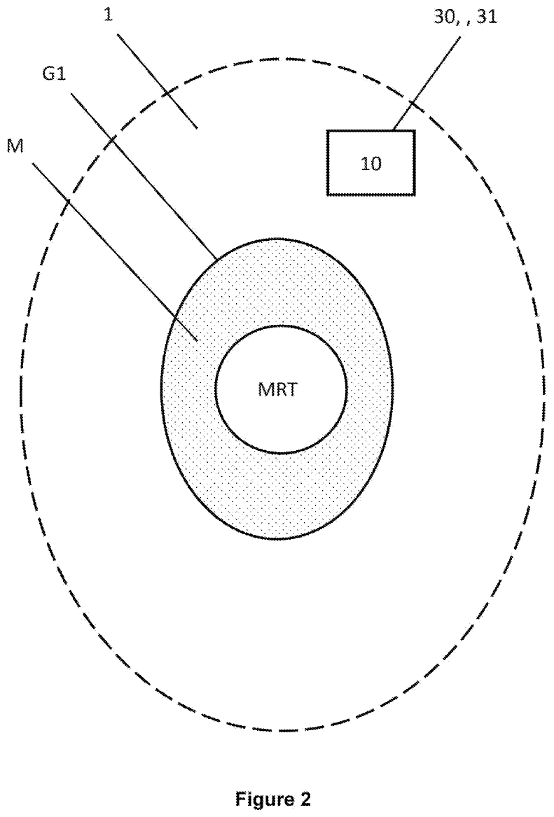

FIG. 2 shows a schematic illustration of the magnetic field M which originates from an MRT.

In the center of the depiction, a magnetized object such as for example a magnetic resonance tomograph (MRT) is schematically shown. A magnetic field M originates from the MRT, which is schematically shown around the MRT as a dotted area in FIG. 2. The magnetic field M which originates from the MRT generally exists permanently. The magnetic flux density of the magnetic field M can be variable, however. The magnetic flux density of the magnetic field M can increase, for example during startup of the MRT. Moreover, the magnetic flux density of the magnetic field M can change during the operation of the MRT.

The identification device 30 can be configured and designed to identify a magnetic field M in that the magnetic flux density or a statistical value of the magnetic flux density, for example a mean value, is above a limiting value G1.

The magnetic field M of the MRT can thus be defined by the limiting value G1. The limiting value G1 can be, for example, above 20 microtesla. 100 microtesla, 500 microtesla, or more can also be provided as the limiting value G1. In preferred embodiments, the limiting value G1 can also be in the millitesla range. For example, the limiting value G1 is in a range from about 20 microtesla (20 μT) to about 5 millitesla (5 mT), preferably in a range from about 500 μT to about 5 mT, particularly preferably in a range from about 1 mT to about 4 mT. In a specific exemplary embodiment, the limiting value G1 is about 3 mT.

The identification device 30 is configured and designed to identify an area in which the values of the magnetic flux density are equal to or less than the limiting value G1 as a noncritical zone 1. In the noncritical zone 1, for example, the magnetic flux density can be from about 0 to about 3 millitesla. In the noncritical zone 1, the magnetic flux density can be, for example, in a range which corresponds to the normal atmosphere or the Earth's magnetic field. In the noncritical zone 1, the ventilator 10 can be operated in the energy-saving mode.

FIG. 2 shows by way of example that the schematically illustrated ventilator 10 is not in the magnetic field M of the MRT.

In the noncritical zone 1, the identification device 30 can, for example, cyclically detect the magnetic flux density. In the noncritical zone 1, the identification device 30 can detect magnetic fields M, for example, cyclically every 1 s or more rarely, in particular about every 2 s or more rarely. The identification device 30 can preferably cyclically detect magnetic fields M about every 4 s, when the ventilator 10 is in the noncritical zone 1.

In a specific exemplary embodiment, the signal device 31 can be activated by the identification device 30 in such a way that an optical signal 33 is output when the identification device 30 identifies that the ventilator 10 is in the noncritical zone 1. The optical signal 33 can then be output, for example, in the form of a continuously lighted green LED light when the ventilator 10 is located in the noncritical zone 1. The optical signal 33 can also be output, for example, in the form of an intermittently flashing green LED signal when the ventilator 10 is located in the noncritical zone 1. Alternatively or additionally, an acoustic signal 35 can be output, for example in the form of an intermittent short single tone, which signals to the user or medical personnel that the ventilator 10 is located in the noncritical zone 1.

In the clinical daily routine, it can occur that the ventilator 10 actively enters the magnetic field of an MRT due to a change of its location. In the clinical daily routine, it can also occur that the ventilator 10 passively enters the magnetic field M due to a change of the magnetic field M which originates from the MRT.

The identification device 30 of the ventilator 10 is configured to detect the magnetic flux density at predefined points in time and preferably in a periodically repeating manner. At least the at least one limiting value G1 can be stored in the identification device 30.

Exceeding the limiting value G1 can signal a physical approach to a magnetized object such as an MRT. Exceeding the limiting value G1 can also signal that an elevated magnetic force originates from a magnetized object such as an MRT.

Reaching and/or falling below the limiting value G1 can signal a physical distancing from a magnetized object such as an MRT. Reaching and/or falling below the limiting value G1 can also signal that a reduced magnetic force originates from a magnetized object such as an MRT.

The identification device 30 can, for example, cyclically detect the magnetic flux density.

If the ventilator 10 is located in the magnetic field M, the identification device 30 can detect magnetic fields M, for example cyclically at least about every 4 s or more frequently, preferably at least about every 500 ms or more frequently, particularly preferably at least about every 200 ms. For example, the identification device 30 can cyclically detect magnetic fields M about every 100 ms when the ventilator 10 is in the magnetic field M.

In a simple exemplary embodiment according to FIG. 2, the ventilator 10 can supply a binary identification with its identification device 30 as to whether or not the ventilator 10 is in a magnetic field M that exceeds a limiting value G1.

In alternative exemplary embodiments, more than one limiting value can be stored in the identification device 30, for example at least two or at least three.

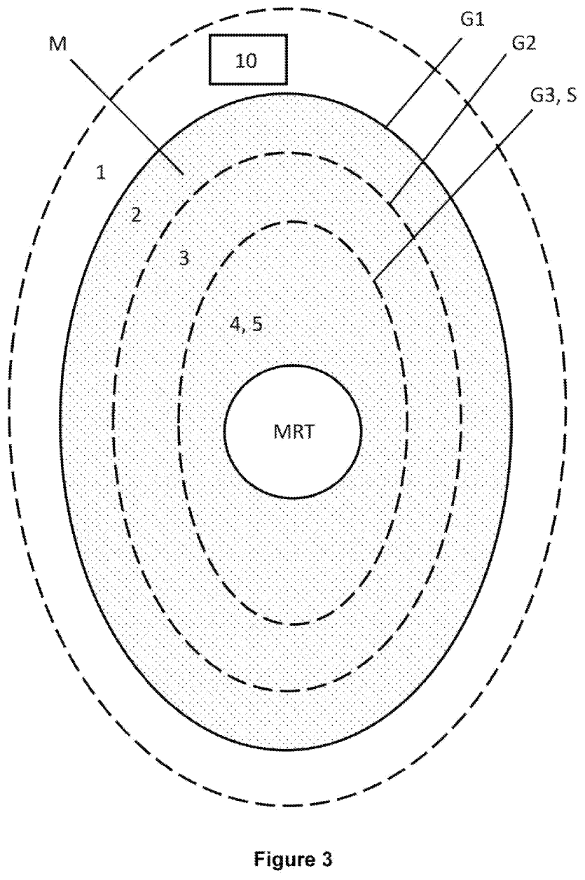

FIG. 3 shows a schematic illustration of different exemplary zones of the magnetic field M which can be identified by the ventilator 10. FIG. 3 shows by way of example that the schematically shown ventilator 10 is not located in the magnetic field M of the MRT.

In the specific exemplary embodiment according to FIG. 3, three limiting values can be stored in the identification device 30 of the ventilator 10, on the basis of which the magnetic field M of the MRT can be divided into different zones. More than three limiting values are also conceivable.

The identification device 30 is configured and designed to identify an area in which the values of the magnetic flux density are equal to or less than the limiting value G1 as a noncritical zone 1. In the advantageous exemplary embodiment according to FIG. 3, a limiting value G3 can additionally define a critical zone 4 and/or a collision zone 4.

The identification device 30 can be configured and designed to identify the critical zone 4 in that the magnetic flux density or a statistical value of the magnetic flux density, for example a mean value, is above the limiting value G3. The limiting value G3 can be, for example, in a range from about 3 mT to about 100 mT, preferably in a range from about 20 mT to about 70 mT. In a specific exemplary embodiment, the limiting value G3 is about 50 millitesla (50 mT). In the critical zone 4, the magnetic flux density can thus be, for example, at least about millitesla (50 mT).

In the critical zone 4, the magnetic flux density can be, for example, in a range in which damage to the ventilator 10 is probable or at least cannot be excluded. The ventilator 10 should be removed immediately from the critical zone 4. Accordingly, components associated with the ventilator 10 should also be removed from the critical zone 4.

As soon the magnetic flux density of the limiting value G3 is exceeded, the identification device 30 can activate the signal device 31. The signal device 31 can be activated in such a way that an optical signal 33 and/or an acoustic signal 35 is output when the identification device 30 identifies that the ventilator 10 is located in the critical zone 4. Preferably, both an optical signal 33 and an acoustic signal 35 are output when the identification device 30 identifies that the ventilator 10 is located in the critical zone 4.

In a specific exemplary embodiment, the optical signal 33 can be output for example in the form of a flashing red LED light when the ventilator 10 is located in the critical zone 4. Alternatively or additionally, the acoustic signal 35 can be output, for example, in the form of a continuous tone.

Alternatively or additionally, a speech output and/or a display in the display device 12 can also be output at the ventilator 10, for example a speech message “Near collision with MRI”.

The optical signal 33 and/or the acoustic signal 35 which is output in the critical zone 4 can be manually reset. The optical signal 33 and/or the acoustic signal 35 which is output in the critical zone 4 can also be automatically reset or changed by the identification device 30 as soon the ventilator 10 leaves the critical zone 4.

In the advantageous exemplary embodiment according to FIG. 3, alternatively or additionally, in addition to the critical zone 4, a collision zone 5 can be defined by the limiting value G3 and/or a threshold value S. The collision zone 5 is preferably defined in that both the limiting value G3 and the threshold value S are exceeded.

The identification device 30 can be configured and designed to identify the collision zone 5 in that the magnetic flux density or a statistical value of the magnetic flux density, for example a mean value, is above the limiting value G3. The limiting value G3 can be, for example, in a range from about 3 mT to about 100 mT, preferably in a range from about 20 mT to about 70 mT. In a specific exemplary embodiment, the limiting value G3 is about 50 millitesla (50 mT).

The identification device 30 can be configured and designed to identify the collision zone 5 in that alternatively or additionally the threshold value S is exceeded. The threshold value S is not a field strength, rather a mathematical value for the exposure counter function 36. Exceeding the threshold value S can signal that the ventilator 10 has been located for too long in the vicinity of a magnetized object, such as an MRT.

Reaching and/or falling below the threshold value can signal a physical moving away from a magnetized object such as an MRT. Reaching and/or falling below the threshold value S can also signal that a reduced magnetic force originates from a magnetized object such as an MRT.

The exposure counter function 36 is configured, if the limiting value G3 is exceeded, to integrate field strength and time up during approach to the MRT (modeling of the heating of inductances) or to integrate them down upon moving away from the field of the MRT (modeling of cooling).

If the exposure counter reaches the threshold value S, a permanent impairment of the ventilator 10 cannot be excluded and submission for checking is requested via a permanent alarm. The threshold value S is in this case not a field strength, rather a mathematical value for the exposure counter function 36.

The identification device 30 is therefore configured and designed to identify an area in which the values of the magnetic flux density are equal to or less than the limiting value G3 and/or the threshold value S is exceeded as the collision zone 5.

In the collision zone 5, the magnetic flux density can be, for example, at least about 50 millitesla (50 mT). In the collision zone 5, the magnetic flux density can be, for example, in a range in which permanent damage to the ventilator 10 and/or components of the ventilator 10 and/or components associated with the ventilator 10 is very probable or at least cannot be excluded.

The ventilator 10 can start and/or continue ventilation in the collision zone 5, but only under a permanent alarm which signals to the user that the ventilator 10 should be disconnected from the patient and must be submitted for service.

As soon the magnetic flux density of the limiting value G3 and/or the threshold value S is exceeded, the identification device 30 can activate the signal device 31.

The signal device 31 can be activated in such a way that an optical signal 33 and/or an acoustic signal 35 is output when the identification device 30 identifies that the ventilator 10 is located in the collision zone 5. Preferably, both an optical signal 33 and an acoustic signal are output when the identification device 30 identifies that the ventilator 10 is located in the collision zone 5.

The activation can take place as soon as the identification device 30 identifies at least one magnetic flux density above the limiting value G3. In some embodiments, it is also provided that the activation of the signal device 31 takes place with a time delay. For example, the signal device 31 can be activated only when the magnetic flux density is above the limiting value G3 more than once. For example, a statistical value of the magnetic flux density, for example a mean value, from the measurement data of multiple points in time can be used to trigger the signal or the alarm. False alarms can thus be avoided. Since the detection of the magnetic flux density preferably takes place in intervals having points in time in the millisecond range, giving the alarm rapidly can still be ensured.

The signal 33, 35, which is generated as a result of an identification of the collision zone 5, can include that a permanent acoustic signal 35 is generated and/or an optical signal 33 is generated, which can be reset only after testing or exchanging critical components.

In a specific exemplary embodiment, the optical signal 33 can be output, for example, in the form of a continuously lighted red LED light when the ventilator 10 is located in the collision zone 5. Alternatively or additionally, the acoustic signal 35 can be output, for example, in the form of a continuous siren tone.

The optical signal 33 and/or the acoustic signal 35 which is output in the collision zone 5 can preferably not be reset manually. The optical signal 33 and/or the acoustic signal 35 which is output in the collision zone 5 can preferably only be reset or changed once the ventilator 10 leaves the collision zone 5 and a test and/or an exchange of critical components has been performed.

In the advantageous exemplary embodiment according to FIG. 3, a limiting value G2 can alternatively or additionally define a warning zone 3.

The identification device 30 can be configured and designed to identify the warning zone 3 in that the magnetic flux density or a statistical value of the magnetic flux density, for example a mean value, is above the limiting value G2. The limiting value G2 can be, for example, in a range from about 3 mT to about 50 mT, preferably in a range from about 10 mT to about 40 mT. In a specific exemplary embodiment, the limiting value G2 is about 20 millitesla (20 mT). In the warning zone 3, the magnetic flux density can therefore be for example at least about 20 mT.

In the warning zone 3, the magnetic flux density can be, for example, in a range which is between the limiting values G2 and G3. The magnetic flux density in the warning zone 3 can therefore be, for example, from about 20 mT to about 50 mT.

In the warning zone 3, the magnetic flux density can be, for example, in a range in which permanent damage to the ventilator 10 is improbable. The warning zone 3 can represent a maneuvering area of the ventilator 10 in which the ventilator 10 can still be operated untested, but a warning with respect to an increased magnetic field is already output. As soon as the magnetic flux density of the limiting value G2 is exceeded, the identification device 30 can activate the signal device 31 in such a way that a warning signal is emitted.

For example, the signal device 31 can be activated in such a way that an optical signal 33 and/or an acoustic signal 35 is output when the identification device 30 identifies that the ventilator 10 is located in the warning zone 3. Such a warning alarm can comprise, for example, that a yellow LED light lights up in flashes and/or an intermittent warning tone is output. Such a warning alarm can notify a user or the clinical personnel that the ventilator 10 is in a maneuvering area in which the magnetic field M is increased.

The optical signal 33 and/or the acoustic signal 35 which is output in the warning zone 3 can be manually reset. The optical signal 33 and/or the acoustic signal 35 which is output in the warning zone 3 can also be automatically reset or changed by the identification device 30 as soon the ventilator 10 leaves the warning zone 3.

Alternatively or additionally, a speech output and/or a display in the display device 12 can also be output on the ventilator 10, for example a speech message: “Distance to MRI too close”.

In one advantageous exemplary embodiment, the limiting value G2 can alternatively or additionally define an operating zone 2. In the operating zone 2, the magnetic flux density can be, for example, in a range which is between the limiting values G2 and G1. The magnetic flux density in the operating zone 2 can therefore be, for example, from about 3 mT to about 20 mT.

In the operating zone 2, the magnetic flux density can be, for example, in a range in which damage to the ventilator 10 due to a magnetic field M is very improbable. The operating zone 2 can represent the recommended area for operation of the ventilator 10. As soon as the magnetic flux density of the limiting value G2 is exceeded, the identification device 30 can activate the signal device 31 in such a way that an operating alarm is emitted.

For example, the signal device 31 can be activated in such a way that an optical signal 33 is output when the identification device 30 identifies that the ventilator 10 is located in the operating zone 2.

In a specific exemplary embodiment, such an operating alarm can comprise that, for example, a green LED light lights up by flashing. Such an operating alarm can notify a user or the clinical personnel that the ventilator 10 is located in the operating zone 2 in which the magnetic field M is only increased in such a way that it very probably does not disturb or damage the ventilator.

In some exemplary embodiments, the ventilator 10 can be configured and designed to identify whether the tesla sensor of the identification device 30 actively detects signals or whether the tesla sensor is inactive. The ventilator 10 can accordingly be configured and designed to identify a system error of the identification device 30. A system error of the identification device 30 can be present when the tesla sensor is inactive or defective or there are other reasons for obstruction in carrying out a measurement of the magnetic field strength.

In a specific exemplary embodiment, such a system error of the tesla sensor can signal to the signal device 31 that an optical signal 33 is generated. For example, a blue LED light can light up by flashing when a system error of the tesla sensor is identified.

Alternatively or additionally, a speech output and/or a display in the display device 12 can also be output in the ventilator 10, for example a speech message: “Error of tesla sensor”.

The optical signals 33 and/or the acoustic signals 35 can be output increasingly more intensely with increasing strength of the magnetic field M. The optical signals 33 can be output, for example, increasingly brighter or clearer with increasing strength of the magnetic field M. The acoustic signals 35 can be, for example, output increasingly louder with increasing strength of the magnetic field M. An exponentially increasing warning can thus be output to the surroundings.

For example, the signal device 31 can be configured in such a way that switching off of the signal 33, 35 in the event of a system error of the tesla sensor can take place only once a testing plan has been processed and/or critical components have been tested or exchanged, since it is not ensured that the ventilator 10 was not operated in impermissible field strengths while the field could not be measured.

A specific exemplary embodiment of a ventilator 10 having an identification device 30 is configured as follows. From exceeding the threshold from the operating zone 2 to the warning zone 3 (green yellow), acoustic signaling begins at an interval of about ½ s. This increases continuously until directly before reaching the threshold from the warning zone 3 to the critical zone 4 (yellow red) to about 1/100 ms. Upon reaching the threshold yellow red, the signaling goes over to a continuous tone. During withdrawal of the ventilator 10 from the field, the signaling takes place in the reverse direction, thus from possibly a continuous tone to increasing interval length with decreasing field.

To achieve the ability to distinguish between the system error (also collision alarm) from the above-mentioned signaling with variable interval lengths, the acoustic signaling of system errors takes place by three short signal tones in rapid sequence with subsequently a somewhat longer pause.

The acoustic signaling of the collision alarm is reset when the ventilator 10 is switched off (delayed by the timeout to identify the missing communication). From this point on, the acoustic signaling again takes place according to field strength.

The optical signaling of the collision alarm is engaged by the blue LED (about 2.5/s), the display of the field strength ranges by the “traffic signal” continues to take place independently thereof. In the power-saving range (ventilator off, field<about 3 mT), the blue LED flashes for energy-saving purposes at the measurement interval (about ¼ s), the green LED continues asynchronously thereto at about ⅓ s. The optical signaling of the collision alarm is not reset by turning off or turning back on the ventilator.

To sum up, the present invention provides:
1. A ventilator, wherein the ventilator comprises an identification device which is configured and designed to identify at least one magnetic field M.
2. The ventilator of item 1, wherein the identification device 30 is at least one sensor or comprises at least one sensor that is configured and designed to detect magnetic flux densities and/or magnetic field strengths to identify the at least one magnetic field M.
3. The ventilator of item 2, wherein the sensor is a magnetic field sensor that is configured and designed to measure the magnetic field M in three axes.
4. The ventilator of any one of the preceding items, wherein at least one limiting value G1 is stored in the identification device the identification device is configured and designed to identify the at least one magnetic field M when the magnetic flux density or a statistical value of the magnetic flux density exceeds a limiting value G1.
5. The ventilator of any one of the preceding items, wherein the identification device is configured and designed to detect a magnetic flux density once or continuously or cyclically at time intervals, the time intervals repeating regularly and/or irregularly.
6. The ventilator of item 5, wherein the time intervals are constant or are ascertained and dynamically adapted in operation of the ventilator.
7. The ventilator of any one of the preceding items, wherein the identification device detects a magnetic flux density cyclically about every 60 seconds or more frequently, preferably about every 10 seconds or more frequently, particularly preferably about every 5 seconds or more frequently.
8. The ventilator of any one of the preceding items, wherein the identification device detects a magnetic flux density about every 4 seconds or more rarely as long as the limiting value G1 is not exceeded.
9. The ventilator of any one of the preceding items, wherein the identification device cyclically detects a magnetic flux density about every 4 seconds or more frequently, particularly preferably about every 1 second or more frequently, particularly preferably about every 500 milliseconds or more frequently, in particular about every 100 milliseconds when the limiting value G1 is exceeded.
10. The ventilator of any one of the preceding items, wherein the identification device detects the magnetic flux density in a manner dynamically adapted to a magnetic field strength when the limiting value G1 is exceeded.
11. The ventilator of any one of the preceding items, wherein the ventilator comprises a signal device which is configured and designed to emit at least one signal, the identification device being configured and designed to control the signal device, and wherein the signal device comprises an optical signal generator and/or an acoustic signal generator.
12. The ventilator as of any one of the preceding items, wherein the identification device is configured and designed to identify an area in which values of a magnetic flux density are equal to or less than a limiting value G1 as a noncritical zone.
13. The ventilator of item 12, wherein the limiting value G1 ranges from about 500 μT to about 5 mT, particularly preferably from about 1 mT to about 4 mT, the limiting value G1 being preferably about 3 mT.
14. The ventilator of any one of items 12 and 13, wherein one or more additional limiting values are stored in the identification device, on the basis of which the identification device identifies two or more zones of the magnetic field M.
15. The ventilator of item 14, wherein a limiting value G3 is stored in the identification device and the identification device is configured and designed to identify an area in which the values of the magnetic flux density are higher than the limiting value G3 as a critical zone and/or collision zone.
16. The ventilator of item 15, wherein the limiting value G3 ranges from about 3 mT to about 100 mT, particularly preferably from about 20 mT to about 70 mT, the limiting value G3 being preferably about 50 mT.
17. The ventilator of any one of items 15 and 16, wherein a threshold value S is stored in the identification device and the identification device is configured and designed to identify an area in which the values of the magnetic flux density are higher than the limiting value G3 and the threshold value S is exceeded as a collision zone.
18. The ventilator of any one of items 15 to 17, wherein a limiting value G2 is stored in the identification device and the identification device is configured and designed to identify an area in which values of a magnetic flux density are higher than the limiting value G2 and equal to or less than the limiting value G3 as a warning zone.
19. The ventilator of item 18, wherein the limiting value G2 ranges from about 3 mT to 50 about mT, particularly preferably from about 10 mT to about 40 mT, the limiting value G2 being preferably about 20 mT.
The ventilator of any one of items 15 to 19, wherein the identification device is configured and designed to identify an area in which the values of the magnetic flux density are higher than a limiting value G1 and equal to or less than the limiting value G2 as an operating zone.
21. The ventilator of any one of the preceding items, wherein the identification device activates the signal device on the basis of the identification of various zones of the magnetic field M in such a way that, depending on a specific zone, individual optical signals and/or acoustic signals are output.
22. The ventilator of item 21, wherein the optical signals and/or the acoustic signals are output increasingly more intensely with increasing strength of the magnetic field M.
23. The ventilator of any one of items 21 and 22, wherein an optical signal and/or an acoustic signal is automatically adapted when the identification device identifies that one of the limiting values G1, G2, G3 is exceeded or has fallen below.
24. The ventilator of any one of items 21 to 23, wherein an optical signal and/or an acoustic signal is automatically adapted when the identification device identifies that the threshold value S is exceeded or has fallen below.
25. The ventilator of any one of items 21 to 24, wherein an optical signal and/or an acoustic signal, which is output when the ventilator is located in a collision zone, can be ended only after critical components of the ventilator have been tested and/or exchanged.

Although the present invention has been described in detail on the basis of the exemplary embodiments, it is evident to a person skilled in the art that the invention is not restricted to these exemplary embodiments. Rather, modifications are possible in such a manner that individual features are omitted or other combinations of the described individual features can be implemented as long as the scope of protection of the appended claims is not departed from. The present disclosure includes all combinations of the presented individual features.

### LIST OF REFERENCE SIGNS

G1 limiting value
G2 limiting value
G3 limiting value
M magnetic field
MRT/MRI magnetic resonance tomograph
S threshold value
1 noncritical zone
2 operating zone
3 warning zone
4 critical zone
5 collision zone
10 ventilator
11 operating device
12 display device
13 interface
14 hose system
15 patient interface
16 fan device
17 drive
18 energy source
19 control device
20 storage device
30 identification device
31 signal device
32 optical signal generator
33 optical signal
34 acoustic signal generator
35 acoustic signal
36 exposure counter function

What is claimed is:

1. A ventilator, wherein the ventilator comprises an identification device which is configured and designed to identify at least one magnetic field M.

2. The ventilator of claim 1, wherein the identification device is at least one sensor or comprises at least one sensor that is configured and designed to detect magnetic flux densities and/or magnetic field strengths to identify the at least one magnetic field M.

3. The ventilator of claim 2, wherein the sensor is a magnetic field sensor that is configured and designed to measure the magnetic field M in three axes.

4. The ventilator of claim 1, wherein at least one limiting value G1 is stored in the identification device, the identification device being configured and designed to identify the at least one magnetic field M when a magnetic flux density or a statistical value of the magnetic flux density exceeds a limiting value G1.

5. The ventilator of claim 1, wherein the identification device is configured and designed to detect a magnetic flux density once or continuously or cyclically at time intervals, the time intervals repeating regularly and/or irregularly.

6. The ventilator of claim 5, wherein the time intervals are constant or are ascertained and dynamically adapted in operation of the ventilator.

7. The ventilator of claim 5, wherein the identification device detects the magnetic flux density cyclically every 60 seconds.

8. The ventilator of claim 4, wherein the identification device detects a magnetic flux density in a manner dynamically adapted to a magnetic field strength when the limiting value G1 is exceeded.

9. The ventilator of claim 1, wherein the ventilator comprises a signal device which is configured and designed to emit at least one signal, the identification device being configured and designed to control the signal device, and wherein the signal device comprises an optical signal generator and/or an acoustic signal generator.

10. The ventilator as of claim 1, wherein the identification device is configured and designed to identify an area in which values of a magnetic flux density are equal to or less than a limiting value G1 as a noncritical zone.

11. The ventilator of claim 10, wherein the limiting value G1 ranges from 500 RT to 5 mT.

12. The ventilator of claim 4, wherein one or more additional limiting values are stored in the identification device, on the basis of which the identification device identifies two or more zones of the magnetic field M.

13. The ventilator of claim 12, wherein a limiting value G3 is stored in the identification device and the identification device is configured and designed to identify an area in which the values of the magnetic flux density are higher than the limiting value G3 as a critical zone and/or collision zone.

14. The ventilator of claim 13, wherein the limiting value G3 ranges from 3 mT to 100 mT.

15. The ventilator of claim 13, wherein a threshold value S is stored in the identification device and the identification device is configured and designed to identify an area in which the values of a magnetic flux density are higher than the limiting value G3 and the threshold value S is exceeded as a collision zone.

16. The ventilator of claim 13, wherein a limiting value G2 is stored in the identification device and the identification device is configured and designed to identify an area in which values of the magnetic flux density are higher than the limiting value G2 and equal to or less than the limiting value G3 as a warning zone.

17. The ventilator of claim 16, wherein the limiting value G2 ranges from 3 mT to 50 mT.

18. The ventilator of claim 16, wherein the identification device is configured and designed to identify an area in which the values of the magnetic flux density are higher than a limiting value G1 and equal to or less than the limiting value G2 as an operating zone.

19. The ventilator of claim 1, wherein the identification device activates a signal device on the basis of the identification of various zones of the magnetic field M in such a way that, depending on a specific zone, individual optical signals and/or acoustic signals are output.

20. The ventilator of claim 19, wherein the optical signals and/or the acoustic signals are output increasingly more intensely with increasing strength of the magnetic field M and/or an optical signal and/or an acoustic signal is automatically adapted when the identification device identifies that one of limiting values G1, G2, G3 is exceeded or has fallen below and/or. an optical signal and/or an acoustic signal is automatically adapted when the identification device identifies that a threshold value S is exceeded or has fallen below and/or an optical signal and/or an acoustic signal, which is output when the ventilator is located in a collision zone, can be ended only after critical components of the ventilator have been tested and/or exchanged.
